(19) United States
(12) Patent Application Publication
(10) Pub. No.: US 2022/0345600 A1
(43) Pub. Date: Oct. 27, 2022

(54) MULTIMODE WAVEGUIDE IMAGING

(71) Applicant: Stichting VU, Amsterdam (NL)

(72) Inventors: Liubov Vladimirovna AMITONOVA, Amsterdam (NL); Johannes Fitzgerald DE BOER, Amsterdam (NL)

(21) Appl. No.: 17/232,760

(22) PCT Filed: Oct. 18, 2019
(86) PCT No.: PCT/NL2019/050686

§ 371 Date: Apr. 16, 2021

(30) Foreign Application Priority Data
Oct. 19, 2018 (NL) 2021837

(51) Int. Cl. H04N 5/225; G02B 23/24; A61B 1/07; A61B 1/313; A61B 1/00; A61B 1/06; A61B 1/04

(52) U.S. Cl. CPC H04N 5/2256; G02B 23/2469; A61B 1/07; A61B 1/3135; A61B 1/00006; A61B 1/0669; A61B 1/0638; A61B 1/042; A61B 1/00009; A61B 1/0605; H04N 2005/2255; A61M 25/065

(57) ABSTRACT

An imaging system (100) comprises a multimode waveguide (Wm) configured to receive input light (Li) from a light source (20) into its proximal side (13p) and output a corresponding speckle pattern (Pn) based on the input light (Li) out of its distal side (13d) for illuminating a sample (S) to be imaged. A single-mode waveguide (Ws) is connected to the multimode waveguide (Wm) for coupling the input light (Li) from the light source (20) to the multimode waveguide (Wm). The multimode waveguide (Wm) has a relatively short length (Zm) and relatively high flexural rigidity (R) for maintaining a unique relation between the input characteristic (λ,A) of the input light (Li) into the multimode waveguide (Wm) and a spatial distribution (Ixy) of the speckle pattern (Pn). The single-mode waveguide (Ws) may be relatively long and flexible (F) for allowing movement of the short rigid multimode waveguide (Wm).

### TECHNICAL FIELD AND BACKGROUND

The present disclosure relates to imaging systems and methods for illuminating samples with speckle patterns generated via a multimode waveguide.

Light microscopy has been a key tool for biological and medical research for many centuries. However, traditional optical imaging is normally possible only up to a few millimeters below the tissue surface, because at greater depths, multiple light scattering may deteriorate the image. To overcome this constraint, endo-microscopy techniques have been developed. In endo-microscopy it is desired to provide high-resolution images in-vivo through a tiny probe inserted into living tissue. Optical endo-microscopy may support functional or structural imaging due to variety of optical contrast mechanisms, such as elastic scattering, fluorescence, Raman scattering etc. Endo-microscopy can be based, e.g., on miniature optical probes, such as fiber bundles, gradient-index (GRIN) lenses and multimode fibers.

The use of fiber bundles may suffer from a relatively low imaging resolution (about 3 μm) dictated by the minimal possible distance between the cores. Miniature GRIN probes typically provide better spatial resolution but may suffer from aberrations and field of view limits: for example, a typical GRIN probe with a diameter of 1 mm has a field of view (FOV) of only about 250 μm. That can make the endoscopic probe diameter significantly larger than the field of view. Multimode fiber based endoscopes can provide high resolution at full FOV but may rely on the use of complex spatial light modulation systems and knowledge of the transfer matrix of the multimode fiber to reconstruct an imaging. Finally, most state-of-the-art techniques of endo-microscopy are based on raster scanning, which becomes slow for a high number of pixels.

Rodriguez-Cobo et al. [Proc. of SPIE Vol. 8413 84131R-1; doi: 10.1117/12.978217] describe speckle characterization in multimode fibers for sensing applications. As explained, in single mode fibers, the core diameter is relatively small (e.g. 10 μm) and the optical signal has an almost constant phase velocity. In multimode fibers, the diameter is much larger (e.g. ≥50 μm) and the guided modes have different phase velocities. In the first case, the projection of the beam at the output of the fiber typically forms a uniform spot of light, while in the second case a granulated pattern of light may be observed. The latter is generally referred to as a ‘speckle pattern’ and may be understood as an interference phenomenon between modes propagated inside of the multimode fiber. It is suggested that the particular input characteristics of the speckle phenomenon obtained in multimode fibers can be used in sensing technology.

WO 2013/144898 A2 describes a multimode waveguide illuminator and imager which relies on a wave front shaping system that acts to compensate for modal scrambling and light dispersion by a flexible multimode waveguide. A first step consists of calibrating the multimode waveguide and a second step consists in projecting a specific pattern on the waveguide proximal side in order to produce the desire light pattern at its distal side. The illumination pattern can be scanned or changed dynamically only by changing the phase pattern projected at the proximal side of the waveguide by a spatial light modulator. The third and last step consists in collecting the optical information, generated by the sample, through the same waveguide in order to form an image. According to the prior art, the flexible multimode waveguide can be inserted in a sample and moved while adapting the calibration.

Unfortunately, known systems may require frequent recalibration to condition the output of the waveguide to a phase pattern projected at the input. Also, spatial light modulator may be cumbersome and it may be difficult to project sufficiently different phase patterns to provide a desired range of illumination patterns. So there remains a need to alleviate disadvantages in the known systems and methods while maintaining at least some of their advantages.

### SUMMARY

Some aspects of the present disclosure relate to an imaging system e.g. used for endo-microscopy. Preferably, the system comprises a multimode waveguide configured to receive input light from a light source into its proximal side and output a corresponding speckle pattern based on the input light out of its distal side for illuminating a sample to be imaged. By keeping the multimode waveguide relatively rigid, a unique relation may be maintained between the input characteristic of the input light into the multimode waveguide and a spatial distribution of the speckle pattern. By keeping the multimode waveguide relatively short, it may be more easy to handle. For example, a single-mode waveguide may be connected to the multimode waveguide for coupling the input light from the light source to the multimode waveguide. By using a single-mode waveguide which has a relatively long length and is flexible compared to the multimode waveguide this may allow movement of the multimode waveguide with respect to the light source without affecting the input characteristic of the input light into the multimode waveguide.

Some aspects may relate to image reconstruction based on speckle patterns generated in a multimode fiber. For example, image reconstruction may include accessing calibration data relating a predetermined set of respective input characteristics such as a variable wavelength of the input light into the multimode waveguide with a corresponding set of respective spatial distributions of speckle patterns out of the multimode waveguide. For example, a set of (spectral) intensity measurements may be received from the sample illuminated by different speckle patterns according to the set of predetermined spatial distributions. The spatially resolved image of the sample may thus be calculated based on the intensity measurements and calibration data.

These and other aspects may provide various advantages in fields such as endo-microscopy. For example, the methods and systems may enable high-speed diffraction-limited imaging at full field of view of a probe which doesn't require complex elements, such as spatial light modulators or knowledge of the transfer matrix of the multimode fiber to reconstruct the image. Some aspect may relate to a combination of compressive sensing with a multimode fiber probe to produce a random basis of speckle patterns, and illuminate the sample with this random but known set of speckle patterns. Then the fluorescent, elastic scattered or Raman scattered response may be collected and the image reconstructed from this response. Optionally optical sectioning is provided by calibrating the system at different working distances. Advantages of the compressive algorithm may include the possibility of lowering the number of measurements by an order of magnitude compared to a point by point raster scan to get an image, which consists of many thousands of pixels. Consequently, it can be more than an order of magnitude faster than any of the conventional raster scanning approaches of endo-microscopy. Moreover, no detailed information of the transmission matrix of the multimode fiber is needed to reconstruct the image.

Compressive imaging endo-microscope may be based on standard multimode fiber and doesn't require the use of spatial light modulator, high NA objectives and/or massive scanning elements. Therefore it can be cheap, simple and easily miniaturized for biomedical applications. The spatial resolution of this new endo-microscope may be determined by the numerical aperture of a fiber probe and can be very high (multimode fibers with NA>0.8 have already been demonstrated). The field of view is only limited by the fiber probe diameter. Due to its simplicity and compactness, the new endo-microscopes can be used for imaging during medical procedures, through a needle core, for instance during placement of epidural anesthesia.

### BRIEF DESCRIPTION OF DRAWINGS

These and other features, aspects, and advantages of the apparatus, systems and methods of the present disclosure will become better understood from the following description, appended claims, and accompanying drawing wherein:

FIG. 1A illustrates an imaging system;

FIG. 1B schematically illustrates an imaging probe head of the imaging system;

FIGS. 2A and 2B illustrate variable light input corresponding speckle patterns;

FIGS. 3A and 3B illustrate embodiments for controlling the speckle patterns;

FIG. 4A illustrates an imaging system with a broadband light source and multi-spectral light detector;

FIG. 4B illustrates an imaging system with a multi-clad fiber to carry and separate signal light from input light;

FIGS. 5A and 5B illustrate calibration and measurement with different wavelengths;

FIGS. 6A-6C illustrate embodiments for coupling light between source/signal fibers and a multimode fiber;

FIGS. 7A and 7B illustrate an embodiment where the multimode waveguide can be inserted and removed from a hollow needle;

FIGS. 8A and 8B illustrate cross-correlation coefficients of speckle patterns as a function of relative wavelength for different multimode fiber lengths;

FIGS. 9 and 10 illustrate images and graphs based on various measurements.

### DESCRIPTION OF EMBODIMENTS

Terminology used for describing particular embodiments is not intended to be limiting of the invention. As used herein, the singular forms “a”, “an” and “the” are intended to include the plural forms as well, unless the context clearly indicates otherwise. The term “and/or” includes any and all combinations of one or more of the associated listed items. It will be understood that the terms “comprises” and/or “comprising” specify the presence of stated features but do not preclude the presence or addition of one or more other features. It will be further understood that when a particular step of a method is referred to as subsequent to another step, it can directly follow said other step or one or more intermediate steps may be carried out before carrying out the particular step, unless specified otherwise. Likewise it will be understood that when a connection between structures or components is described, this connection may be established directly or through intermediate structures or components unless specified otherwise.

The invention is described more fully hereinafter with reference to the accompanying drawings, in which embodiments of the invention are shown. In the drawings, the absolute and relative sizes of systems, components, layers, and regions may be exaggerated for clarity. Embodiments may be described with reference to schematic and/or cross-section illustrations of possibly idealized embodiments and intermediate structures of the invention. In the description and drawings, like numbers refer to like elements throughout. Relative terms as well as derivatives thereof should be construed to refer to the orientation as then described or as shown in the drawing under discussion. These relative terms are for convenience of description and do not require that the system be constructed or operated in a particular orientation unless stated otherwise.

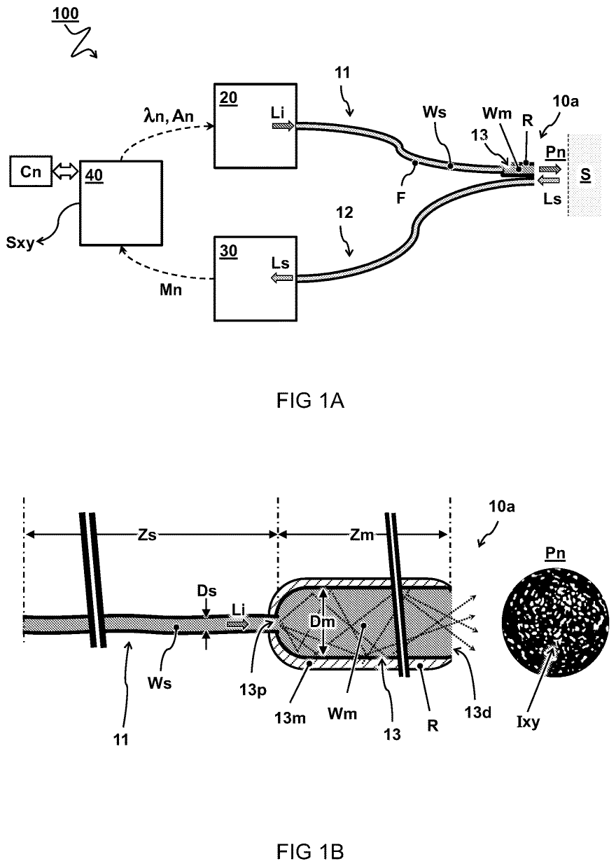

FIG. 1A illustrates an imaging system 100. FIG. 1B schematically illustrates an imaging probe head 10a of the imaging system 100;

In some embodiments, e.g. as shown, the system comprises a multimode waveguide “Wm”. The multimode waveguide may be configured to receive input light “Li”, e.g. from a light source 20, into its proximal side 13p, and output a corresponding speckle pattern “Pn” based on the input light “Li” out of its distal side 13d. The speckle pattern can be used for illuminating a sample “S” to be imaged. Without being bound by theory, the speckle pattern is generally understood as an intensity pattern produced by mutual interference of a set of wavefronts. For example, light passing a multimode waveguide may experience interference between different modes (mode interference).

Typically multimode interference does not occur in a single mode fiber because typically—as the name says—there is only a single mode, i.e. the single mode waveguide is preferably configured to transmit only one mode. In principle, any waveguide configured to support a single mode of the input light may be considered a single mode waveguide. While the single or low mode waveguide may in principle allow additional modes, e.g. at very different wavelengths, the system or light source, is preferably configured to use only the preferred single mode in the measurements to prevent uncontrolled mode interference before the light enters the multimode waveguide.

Preferably, the multimode waveguide “Wm” has a relatively short length “Zm” and/or a relatively high flexural rigidity “R” for maintaining a unique relation between the input characteristic of the input light “Li” into the multimode waveguide “Wm” and a spatial distribution “Ixy” of the speckle pattern “Pn”. For example, the input characteristic may include a wavelength “λn” of the light and/or a spatial distribution “An” of the input light entering the multimode waveguide

In some embodiments, e.g. as shown, the system comprises a single-mode waveguide “Ws” connected to the multimode waveguide “Wm” for coupling the input light “Li” from the light source 20 to the multimode waveguide “Wm”. Preferably, the single-mode waveguide “Ws” has a relatively long length “Zs” and/or is relatively flexible F (compared to the multimode waveguide “Wm”). This may allow movement of the short rigid multimode waveguide “Wm” with respect to the light source 20 without affecting the input characteristic of the input light “Li” into the multimode waveguide “Wm”.

In some embodiments, the multimode waveguide “Wm” has a flexural rigidity much higher than that of the single-mode waveguide “Ws”, e.g. by at least a factor two, three, five, ten, twenty, fifty, hundred, or more. The more rigid the multimode waveguide “Wm”, the better the correlation between the input light “Li” and corresponding speckle pattern “Pn” is maintained. The more flexible the single-mode waveguide “Ws”, the easier it is to freely move around the rigid end formed by the multimode waveguide “Wm”

In a preferred embodiment, e.g. as shown, the multimode waveguide “Wm” is formed by a multimode optical fiber 13. In some embodiments, the multimode optical fiber may be held fixated by a rigid mantle 13m. The mantle 13m may providing the high flexural rigidity “R” for substantially preventing any bending of the multimode waveguide “Wm” which may otherwise destroy the correlation between the input characteristic of the input light “Li” and corresponding speckle pattern “Pn”. In another or further preferred embodiment, the single-mode waveguide “Ws” comprises a single-mode optical fiber 11. Typically, the single-mode optical fiber 11 may be substantially free of the rigid mantle 13m, as shown.

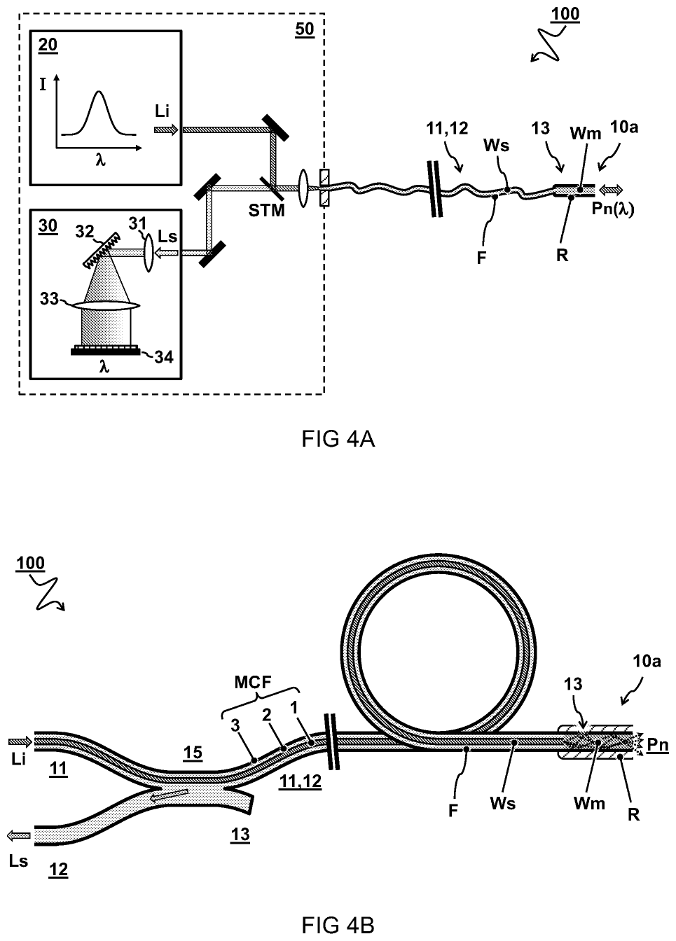

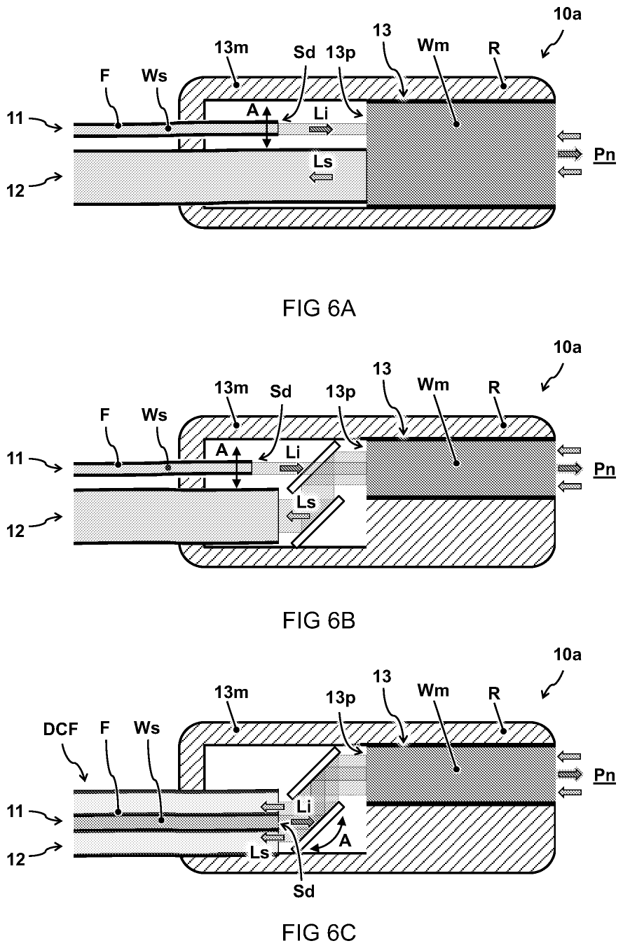

In some embodiments, the rigid multimode waveguide “Wm” forms an imaging probe head 10a connected or connectable to the rest of the system, e.g. light source 20 and/or detector 30, via the flexible connection provided by the single-mode waveguide “Ws”. Preferably, signal light “Ls” coming back from the sample “S” is guided to the detector 30 via a (flexible) multimode waveguide. This can be a separate fiber 12 near the sample location, as shown in FIG. 1A, More preferably, the flexible multimode waveguide guiding the light back is integrated with the flexible multimode waveguide, e.g. as one or more adjacent connected fibers (bundle), or more preferably as a mantle surrounding the single mode fiber to form a double (or multi) clad fiber (e.g. shown in FIGS. 4B and 6C).

In one embodiment, the multimode waveguide “Wm” has a relatively short length “Zm” of less than twenty centimeters, preferably between one and fifteen centimeters, more preferably between two and ten centimeters, most preferably, between three and seven centimeters. In some embodiments, the distal side 13d of the multimode waveguide “Wm” is formed by a roughened scattering surface instead of the typical smooth fiber facet. Roughening the fiber output facet may further improve the generation of uncorrelated speckle patterns. For example, this may allow minimum variation of wavelength at the input to produce different speckle patterns also using relatively short multimode waveguides. Alternatively, or in addition, large variation of wavelengths at the input may allow very short multimode waveguides, e.g. less than five centimeter, less than one centimeter, or less even less than one millimeter. Preferably, the rigid multimode waveguide “Wm” is as short as possible to make it more easy to handle in tight spaces, while still providing sufficiently different speckle patterns for the range of different input characteristics. The length “Zs” of the single-mode waveguide “Ws” can be much higher than the length “Zm” of the multimode waveguide “Wm”, e.g. longer by at least a factor two, three, five, ten, twenty, fifty, hundred, or more. For example, the single-mode waveguide “Ws” can be one or several meters in length.

In one embodiment, e.g. as shown, the system comprises the light source 20 to generate the input light “Li”. As shown, the single-mode waveguide “Ws” can be connected to a fixed output of the light source 20 in a light path between the light source 20 and the multimode waveguide “Wm”. In another or further embodiment, e.g. as shown, the system comprises a light detector 30 configured to determine an intensity measurement “Mn” of a light signal “Ls” from the sample “S” resulting from the illumination by the speckle pattern “Pn”. It will be appreciated that the intensity measurement “Mn” need not be spatially resolved, so the light signal “Ls” can be easily transferred through the same or separate waveguides. In some preferred embodiments, the system comprises optical fibers 11,12,13 forming respective waveguides to transfer the input light “Li” from the light source 20 to the sample “S” and/or transfer the signal light “Ls” from the sample “S” to a light detector 30.

In one embodiment, the single-mode waveguide “Ws” is formed by a single-mode optical fiber 11 having a single-mode waveguide diameter “Ds”, and the multimode waveguide “Wm” is formed by a multimode optical fiber 13 having a multimode waveguide diameter “Dm”. Typically, The multimode waveguide diameter “Dm” is larger than the single-mode waveguide diameter “Ds”, e.g. by a least a factor two, at least a factor three, four, or five, e.g. up to a factor ten or more larger.

In one embodiment, e.g. as shown, the system comprises a controller 40. In some embodiments, the controller may be configured and/or programmed to access calibration data “Cn”. For example, the calibration data may be stored on a computer readable medium which may be part of, or otherwise accessible to the controller 40. In some embodiments, the calibration data may provided relations between a predetermined set of respective input characteristics λn,An of the input light “Li” into the multimode waveguide “Wm” and a corresponding set of respective spatial distributions Ixy of speckle patterns “Pn” out of the multimode waveguide “Wm”. In other or further embodiments, the same or other controller may receive a set of spatially unresolved intensity measurements “Mn” of light signals “Ls” from the sample “S” illuminated by different speckle patterns “Pn” according to the set of predetermined spatial distributions Ixy. In other or further embodiments, the same or other controller may calculate a spatially resolved image “Sxy” of the sample “S” based on the intensity measurements “Mn” and calibration data “Cn”. For example, a compressive sensing algorithm and/or neural network may be used as described herein.

Some aspects of the present disclosure may be embodied as method. In some embodiments, the method comprises receiving calibration data “Cn” relating a set of wavelengths “A” or spatial distributions “A” of input light “Li” into a multimode waveguide “Wm” with a corresponding set of respective spatial distributions Ixy of speckle patterns “Pn” out of the multimode waveguide “Wm”. In other or further embodiments, the method comprises receiving a set of spectrally resolved intensity measurements “Mn” of a light signal “Ls” from a sample “S” illuminated by different speckle patterns “Pn” according to the set of predetermined spatial distributions Ixy.

In some embodiments, the method comprises calculating a spatially resolved image “Sxy” of the sample “S” based on the intensity measurements “Mn” and calibration data “Cn”. The method may be performed e.g. in combination with the rigid multimode waveguide “Wm”. Alternatively, the method may be used in combination with a flexible multimode waveguide “Wm” as long as the calibration is still valid, e.g. the multimode waveguide “Wm” is not substantially bent between the calibration and measurement, and/or it is bent back to have the same configuration (e.g. inserted back into the same rigid mantle 13m).

In a preferred embodiment, the calculation of the spatially resolved image “Sxy” comprises application of a compressive sensing (CS) algorithm which uses a sparsity property of the image to be reconstructed, e.g. the reconstructed image should be sparse in some basis (typically in wavelet basis). This may allow to significantly (more than ten times) increase imaging as well as pre-calibration speed, which is important in any life science and medical applications. Some aspects such as algorithms and/or calibration data may also be embodied as a non-transitory computer readable medium. For example, the medium may storing program instructions or data which when executed and/or accessed by a computer cause the computer to perform the methods as described herein and/or form at least part of an imaging system (e.g. the controller 40) as described.

Also other functions or algorithms such as neural networks may be used for reconstructing the image. For example, a set of known light inputs and speckle pattern outputs may be used to train a neural network. In a sense the coefficients, e.g. weights of the trained neural network, may act as calibration data where the image is calculated based on the coefficients. In principle the network could also be trained (or re-trained) using only the known light inputs and the measured signals at the detector, e.g. assuming some constraint on the images to be reconstructed. For example, the known light input and corresponding signal measurement at the detector may be inputs in a (deep learning) neural network, where deviation from the constraint is used as error or penalty in the training. For example, an image constraint may include a compressibility or entropy of the reconstructed image, where it is assumed that the image to be reconstructed is not completely random, In some embodiments, the training may be continuous, e.g. while performing the actual measurements the network is trained using a set of the latest measurements.

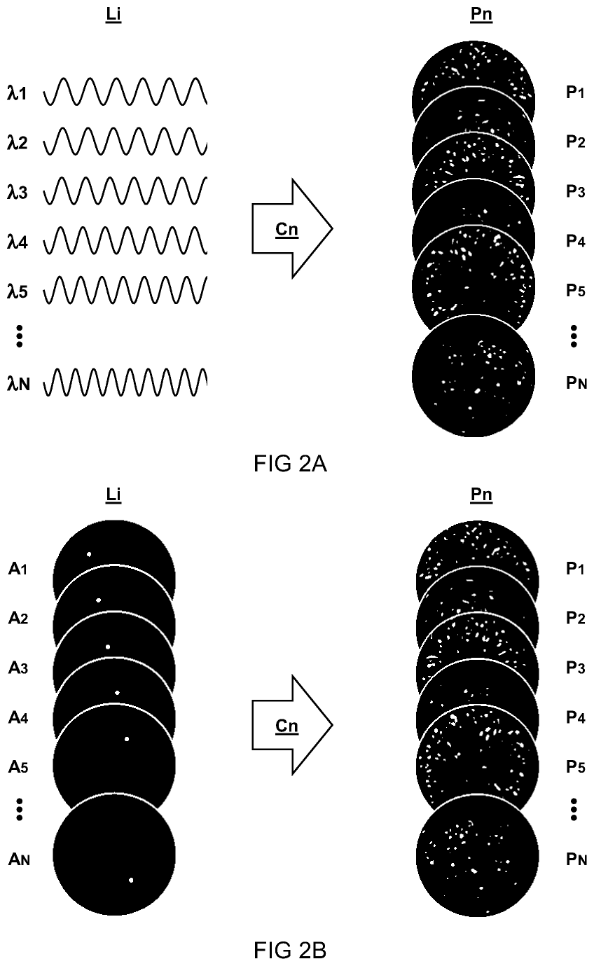

FIGS. 2A and 2B illustrate some of the possible variable light input Li received at a proximal side of a multimode waveguide, and corresponding light output forming different speckle patterns “Pn” at a distal side of the multimode waveguide;

In some embodiments, e.g. as shown in FIG. 2A, the predetermined set of input characteristics comprises a set of different wavelengths “λ” of the input light “Li”. In some embodiments, a controller is configured to reproducibly control the input characteristic “λ” and/or “A” of the input light “Li” into the proximal side of the multimode waveguide “Wm” to uniquely relate the controlled input characteristic “λ” and/or “A” to the spatial distribution “Ixy” of the corresponding speckle pattern “Pn” out of the distal side 13d. For example, the controller is configured to control the light source 20 to sequentially produce a set of different wavelengths “λN” of the input light “Li” at the proximal side 13p of the multimode waveguide “Wm”. It will be appreciated that control of the light source may be relatively easy, e.g. not requiring any mechanical elements such as micro-mirrors. In some embodiments, the calibration data may include normalization of the measured signal for different wavelengths. For example, in a fluorescence measurement, the amount of fluorescent light from the sample may depend on the wavelength of the input light. In other types of measurements, e.g. elastic scattering, the effect of different wavelength on the amount of reflection may be negligible.

In other or further embodiments, e.g. as shown in FIG. 2B, the predetermined set of input characteristics comprises a set of different spatial distributions An of the input light “Li” into the proximal side 13p of the multimode waveguide “Wm”. For example, a controller is configured to sequentially produce a set of different spatial distributions An of the input light “Li” at the proximal side 13p of the multimode waveguide “Wm”. Also combinations of different wavelengths and spatial distributions are possible. Also other variations of the input characteristic of the input light “Li” may be envisioned, e.g. different polarizations, phases, angles, et cetera.

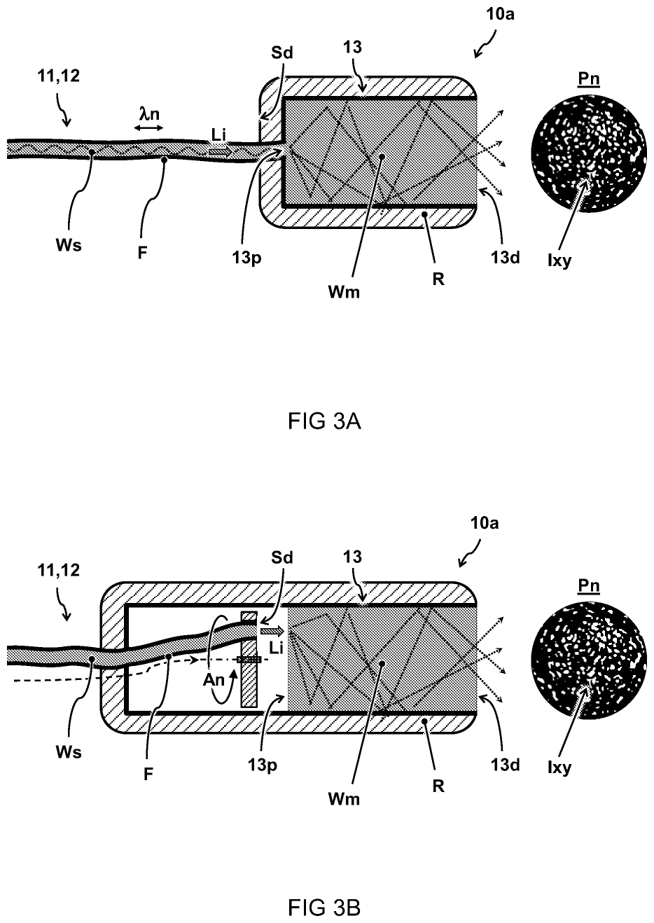

FIGS. 3A and 3B illustrate embodiments for controlling a speckle pattern “Pn” generated in a multimode waveguide “Ws”.

In one embodiment, as shown in FIG. 3A, an output of a single mode fiber 11 forming the single-mode waveguide “Ws” is fused to proximal side 13p of the multimode waveguide “Wm” formed by a multimode optical fiber 13. This may fixate the position and/or angle at which the input light “Li” enters the multimode waveguide “Wm”. This embodiment may be combined e.g. with a variable wavelength “λ” of the input light “Li”. Preferably, a refractive index of material forming the multimode waveguide

“Wm” is the same or similar as that of the single-mode waveguide “Ws”, e.g. within ten percent, preferably within five or one percent, most preferably the same material. The more similar the refractive index, the less reflection may occur at an interface between the single-mode waveguide “Ws” and the multimode waveguide “Wm”.

In another or further embodiment, a position of an end of the single-mode waveguide “Ws”, e.g. optical fiber, is varied with respect to the proximal side 13p of the multimode waveguide “Wm” to provide a set of different input characteristics “An”. For example, the fiber ending is rotated, reciprocated, and/or vibrated, e.g. by a micro motor. Alternatively, or in addition, the light from the single-mode waveguide “Ws” may pass via an optical element, e.g. micro-mirror, which may similarly control the input characteristic “An” (e.g. illustrated in FIG. 6C). A preferred step between different positions of the light spot emanated from the source fiber 11 onto the multimode optical fiber 13 may depend on the wavelength “λ” of the source light and/or the numerical aperture of the multimode waveguide “Wm”. To create sufficiently different (uncorrelated) speckle patterns, preferably, the step is at least λ/2NA.

FIG. 4A illustrates an embodiment using a broadband light source 20 and multi-spectral light detector 30.

In some embodiments, e.g. as shown, the system comprises a broad band light source 20 configured to generate the input light “Li” over a range of different wavelengths “λ”. In other or further embodiments, the system comprises a light detector 30 with a spectral resolving element 32 for measuring an intensity of the light signal “Ls” as a function of wavelength “λ”. In other or further embodiments, the light detector 30 comprises a light sensor 34 with a plurality of sensor elements for simultaneously measuring spectral intensities of the light signal “Ls”.

In a preferred embodiment, the controller is configured to calculate the spatially resolved image based on one or more shots of the broad band light source 20 and corresponding measurements of the spectral intensities of the light signal “Ls”. It will be appreciated that this may allow very fast image acquisition, where essentially a set of measured spectral intensities is converted into a spatial image using calibration data linking the spectral components to respective spatial distributions of the corresponding speckle patterns. In some cases, an intensity of the input light “Li” may vary for different wavelengths and the corresponding light signal “Ls” at that wavelength may be normalized accordingly. For example, a spectrum of (part of) the input light “Li” is simultaneously or sequentially measured for normalization, optionally using the same light detector 30. Alternative to using broad band shots of light, the light source 20 may be controlled to scan the wavelength of the input light “Li”. This may simplify the light detector 30, e.g. dispensing the spectral resolving element 32 and needing only a single light intensity sensor.

In some embodiments, e.g. as shown, in FIG. 4A, the input light “Li” is separated from the returning light signal “Ls” using a semi-transparent mirror “STM”. For example, the semi-transparent mirror TM may pass fifty percent of the light. For some applications where the light signal “Ls” has a substantially different wavelength from the input light “Li”, a dichroic mirror may be used to more efficiently separate the light. For example, this may be applicable in fluorescence measurements.

FIG. 4B illustrates an embodiment with a multi-clad fiber to carry and separate the returning signal light “Ls” from the input light “Li”. In a preferred embodiments, e.g. as shown, the single-mode waveguide “Ws” is part of a multi-clad fiber, e.g. double clad fiber “DCF”. Most preferably, the multi-clad fiber is applied in combination with a broadband light source such as shown in FIG. 4A e.g. allowing easy separation of the input light “Li” and resulting light signal “Ls” particularly when these may have the same or similar wavelength.

In some embodiments, the system comprises a multi-clad fiber formed by at least a fiber core 1 with a first fiber cladding 2 around the fiber core 1, and a second fiber cladding 3 surrounding the first fiber cladding 2. Advantageously, the fiber core 1 may form the single-mode waveguide “Ws” for the input light “Li” and the first fiber cladding 2 may form a return path for the measured light signal “Ls”. In one embodiment, e.g. as shown, both the fiber core 1 and first fiber cladding 2 are connected to couple the input light “Li” into, and the signal light “Ls” out of, the multimode optical fiber 13. For example, the first fiber cladding 2 forms an inner cladding of the double-clad fiber surrounded by a second fiber cladding 3 forming an outer cladding of the double-clad fiber DCF.

In a preferred embodiment, the system comprises a fiber coupler 15 so separate the input light “Li” from the light signal “Ls”. In one embodiment, e.g. as shown, the fiber coupler 15 is an asymmetric multi-clad fiber coupler. For example, the fiber core 1 extends through the coupler 15 between the source fiber 11 connected to the light source (not shown here) and the multimode optical fiber 13 at the imaging probe head 10a. For example, the first fiber cladding 2 is fused with the signal fiber 12 connected to the light detector (not shown here). For example, the first fiber cladding 2 is configured to collect signal light “Ls” from the illuminated sample region S via the multimode optical fiber 13 and transmit at least some of the collected signal light “Ls” via the fiber coupler 15 into the signal fiber 12.

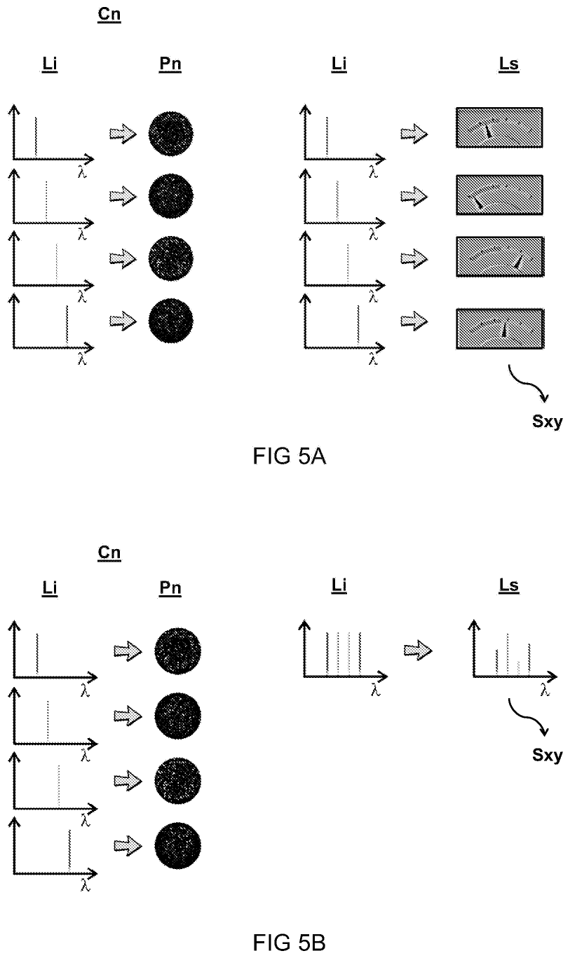

FIGS. 5A and 5B illustrate calibration and measurement with different wavelengths of the input light “Li” being scanned or applied as broadband shots, respectively. For both embodiments, calibration data “Cn” may be obtained e.g. by scanning the wavelength “λ” of the light source and measuring the corresponding speckle patterns “Pn”, e.g. using a camera or pixel array. Then the measurements can be done. For the embodiment of FIG. 5A, the measurements may simply comprise repeating the wavelength scan and measuring a sequence of intensities of the corresponding light signal “Ls”. The spatially resolved image “Sxy” may then be reconstructed e.g. from solving an optimization problem, e.g. compressive sensing algorithm. The embodiment of FIG. 5B may use a similar calculation, but with all spectral intensities measured simultaneously. Similar calibration and reconstruction may also be performed for other types of input characteristics.

In a preferred embodiment, image reconstruction uses compressive sensing (also known as compressive sampling, or sparse sampling). This may be considered a signal processing technique for efficiently acquiring and reconstructing a signal, by finding solutions to underdetermined linear systems. Through optimization, the sparsity of a signal can be exploited to recover it from far fewer samples than required by the Shannon-Nyquist sampling theorem. One condition for recovery may be referred to as “sparsity”. For example, this can be achieved if the signal is sparse in some domain. Another condition may be referred to as “incoherence” which is applied through the isometric property which is sufficient for sparse signals.

FIGS. 6A-6C illustrate various embodiments for coupling light between source/signal fibers 11,12 and multimode fiber 13.

In a preferred embodiment, the signal fiber 12 for returning the light signal “Ls” is also a multimode fiber, having the same or different, e.g. lower, diameter compared to the multimode optical fiber 13 producing the speckle pattern “Pn”. In some embodiment, the single mode source fiber 11 and the returning signal fiber 12 form a single bundle. This may allow moving the imaging probe head 10a with only a single bundle attached. In some embodiments, the source fiber 11 and the signal fiber 12 are both connected to the probe head 10a formed by a rigid mantle 13m housing the multimode optical fiber 13. In some embodiments, the signal fiber 12 is directly in contact with a sample.

In some embodiments, the rigid mantle 13m may house further optical components, e.g. mirrors (shown in FIGS. 6B and 6C) and/or lenses (not shown). For example, miniature microfabricated micro-electromechanical systems (MEMS) can be used. In some embodiments, e.g. as shown in FIG. 6A, a fiber ending of the source fiber 11 may be moved by a micro-motor (not shown) which can be housed in the rigid mantle 13m. In some embodiments, e.g. as shown in FIG. 6B, the imaging probe head 10a may comprise semi-transparent and/or dichroic mirrors to separate a light path with input light “Li” from a light path with light signal “Ls”. Also other elements such as polarizers may be used. In some embodiments, the ends of the source and signal fibers 11,12 may be kept stationary while a reciprocating mirror controls an input characteristic A of the input light “Li”. Of course many variations of the embodiments described herein can be envisioned, using different combinations of elements.

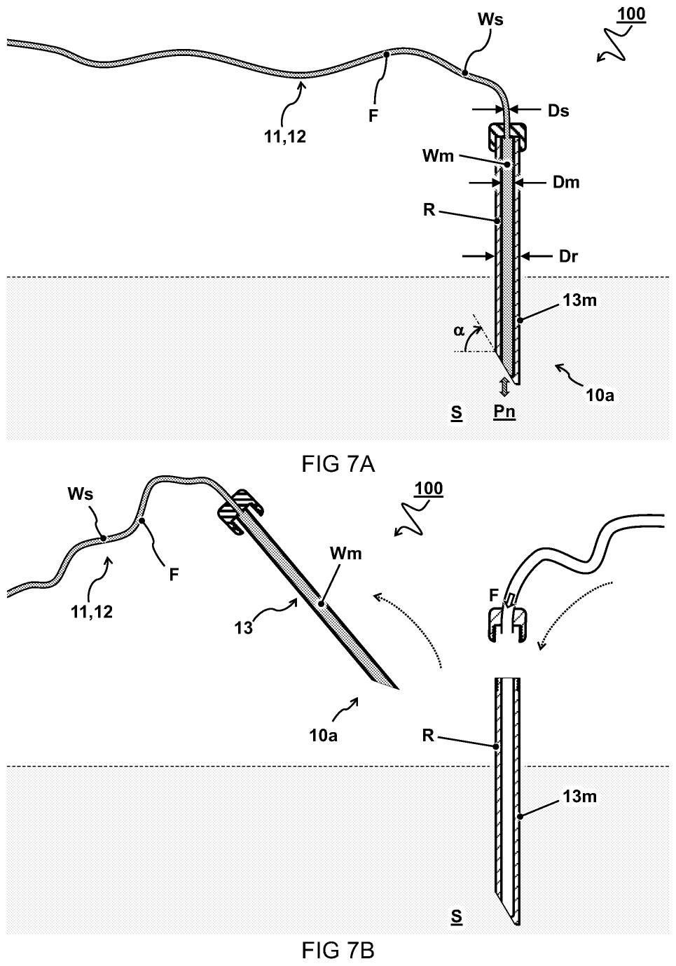

FIGS. 7A and 7B illustrate an embodiment where the multimode waveguide “Wm” can be inserted in a hollow needle. For example, the waveguide “Wm” may be integrated or separable from the needle. In one embodiment, imaging system is used to provide images of the light at the tip of the needle, while the needle is inserted into the sample “S”. This may e.g. aid in the placement of a hypodermic needle. In some embodiments, the multimode waveguide “Wm” may be removed from the hollow needle so the needle may be connected for the administration of fluid into the sample, e.g. medicine and/or anesthetic.

In one embodiment, the hollow needle may substantially form the rigid mantle 13m lending its rigidity to the multimode waveguide “Wm”. In another or further embodiment, the multimode waveguide “Wm” may itself be relatively rigid without the needle so the calibration is not affected when the fiber is removed from the needle. Preferably, the needle and/or waveguide comprises facet arranged at an angle α, e.g. between thirty and seventy degrees, preferably forty-five degrees. In other embodiments, the angle may be lower or there is no angle (α equal zero degrees)

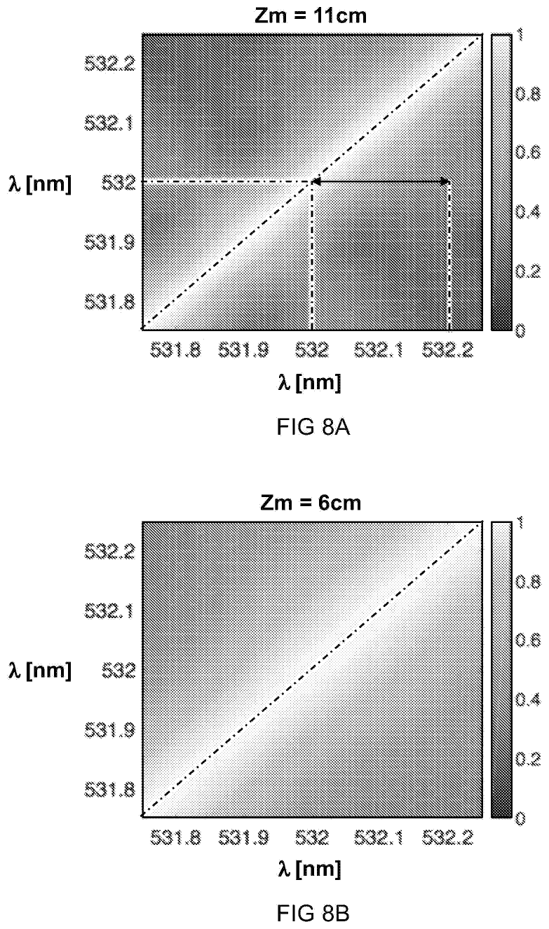

FIGS. 8A and 8B illustrate cross-correlation coefficients of speckle patterns as a function of relative wavelength “λ” for different multimode fiber lengths Zm. Preferably, the set of speckle patterns comprises pseudo-random variable spatial distributions Ixy which are (as much as possible) uncorrelated to each other, e.g. having a correlation “r” less than 0.5, less than 0.2, or less than 0.1. In this case, appropriate decorrelation of speckle patterns may happen at 0.2 nm shift for MM fiber length “Zm”=11 cm, and at 0.4 nm shift for MM fiber length “Zm”=6 cm. So the shift in wavelength “λ” for achieving different speckle patterns may be relatively small. The more uncorrelated the speckle patterns, the less patterns may be needed to reconstruct an image.

It will be appreciated that the present disclosure may provide various advantages in the field endo-microscopy such as compressive multimode fiber imaging. As described herein, the speckle patterns generated in a multimode fiber may represent an excellent basis for compressive sensing. So high-resolution compressive imaging through a fiber probe may be enabled with a total number of measurements much less than would be needed for the standard raster scanning approach to endo-microscopy. Moreover, it will be appreciated that the inherent optical sectioning of a multimode fiber can help to overcome a problem of compressive sensing and can be used for imaging of bulk structures. Compressive multimode fiber imaging, as described herein, does not rely on complex wavefront shaping and can significantly increase pre-calibration and imaging speed, creating advantages for endo-microscopy.

Endoscopy is a key technology for minimally-invasive optical access to deep tissues in living animals. New avenues in endo-microscopy may open up by the emergence of complex wavefront shaping, a method of light control in highly scattering materials. Wavefront shaping allows the use of standard multimode fiber probes as an imaging device. So, multimode fibers may be considered promising tool e.g. for in vivo endo-microscopy. The spatial resolution of multimode-fiber imaging may be determined by the fiber probe's numerical aperture and can be much better than the resolution of conventional fiber-bundle endoscopes. Moreover, a step-index multimode fiber may support a significantly higher number of modes than a fiber bundle, GRIN lens or multicore fiber with the same diameter. So multimode fibers can transfer information at a higher density.

A multimode-fiber-based imaging systems may exploit the idea of conventional scanning fluorescence microscopy. The object on the fiber output facet is thus reconstructed by sequential scanning of each image pixel with the focal points created during the pre-calibration procedure via wavefront shaping. A total fluorescent signal for each pixel is collected and guided back to the registration system through the same fiber.

However, state-of-the-art multimode fiber endo-microscopy may still have limitations. Firstly, the imaging process may takes more time than in standard scanning microscopy because typically a galvo mirror system is be replaced by a much slower spatial light modulator (SLM). The sampling rate may be determined by the desired spatial resolution and must follow the Nyquist criterium. As a result, N≈2N_{modes} measurements are needed for every frame, where N is the total number of pixels in the object image and N_{modes} is the total number of fiber-guided modes. Secondly, the pre-calibration step may need a high number of camera frames to be acquired. For aberration-free imaging with a single polarization input state the number of segments on the SLM is preferably not less than N_{modes}/2. As a result, the number of pre-calibration measurements is typically N₁≥1.5N_{modes}, because at least three phase steps per segment are needed for wavefront-shaping. Finally, typical multimode fiber endo-microscopy may rely on the use of an SLM—a complex and expensive device not common in conventional microscopy.

Aspects of the present disclosure provide a new concept of multimode fiber endo-microscopy: compressive multimode fiber imaging. The approach may provide high-resolution imaging at much higher speed and doesn't require a complex wavefront shaping setup and expensive SLM. In some embodiments, the compressive sensing approach can be combined with a multimode fiber endoscope, which produces a random basis of speckle patterns, collects the fluorescent response and provides optical sectioning by removing the background in case of bulk samples.

Compressive sensing is a novel imaging paradigm that goes against a common view in data acquisition. It relies on the fact that most images have a mathematical property, which is called “sparsity”. This idea underlies most modern lossy codecs such as JPEG-2000. Compressive imaging may implement such compression already at the signal acquisition stage. The image data that would be discarded in compression is never even measured, leading to a significant speed-up of the imaging process.

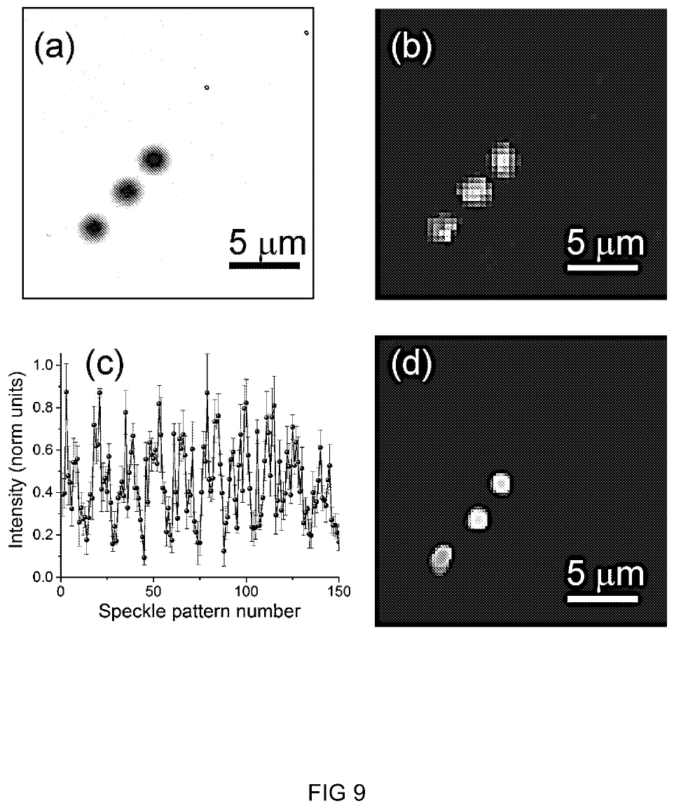

FIG. 9 illustrates an example experiment of multimode-fiber-based imaging of fluorescent spheres. (a) Reference bright-field camera image. (b) Raster scan fluorescent imaging through a multimode fiber via wavefront shaping. (c-d) Compressive multimode fiber imaging: (c) raw data averaged over three measurements after background subtraction and (d) retrieved image using a well-known procedure as the solution to the l₁ minimization problem. Scale bars are 5 μm.

In a first set of measurements, the complex wavefront shaping algorithm was used to create a tightly focused spots on the fiber output. The time required for the optimization procedure was limited by the frame rate of the camera needed for simultaneous optimization of different points. In our experiments, we used a high-speed camera and the whole optimization took 2700 frames and 7.8 seconds. The full width at half maximum (FWHM) of the created foci was 1.14±0.07 μm and agreed perfectly with the diffraction limit of the fiber probe (1.2 μm). The phase masks corresponding to focal spots at a different positions on the output fiber facet were calculated and stored.

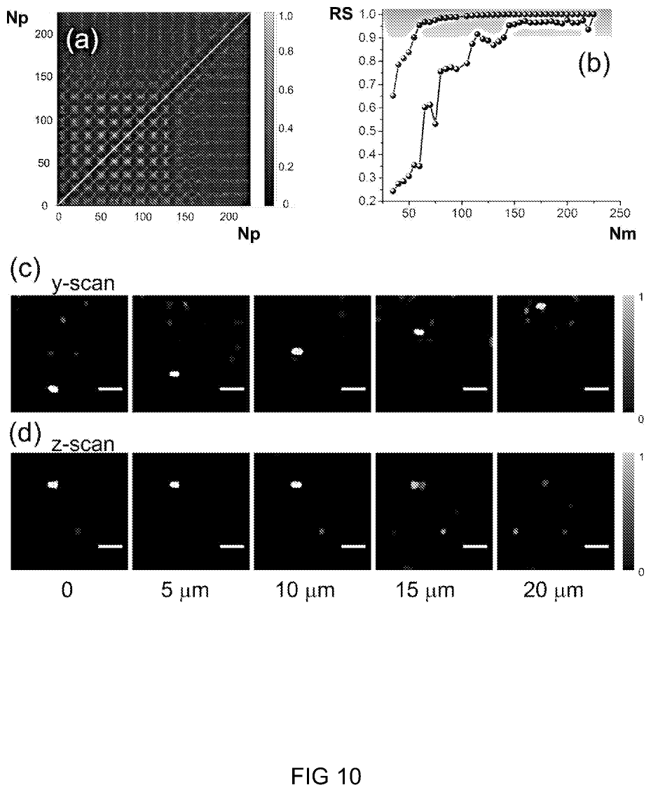

After the wavefront shaping procedure, the sample was placed against the output facet of the multimode fiber. The camera was used to record a bright-field image of the sample, which is presented in FIG. 9(a) for reference. After that, the pre-calibration part was not used anymore. The sample image was acquired in the endoscopy configuration by sequentially applying the recorded phase masks and detecting the total fluorescent signal. As a result, the pixel-by-pixel image of the sample was reconstructed. The result is presented in FIG. 10(b). As shown, there is an excellent agreement between the bright-field reference sample image in FIG. 9(a) and the image recorded through the MM fiber in FIG. 9(b).

In a second set of experiments, we implemented the compressive sensing approach to MM fiber imaging. In the embodiments described here, a digital micro-mirror device (DMD) provided only for amplitude modulation. Of course also other ways of varying the input can be used as described herein. In one embodiment, a pre-calibration procedure is used comprising the recording of speckle patterns on the output facet of the multimode fiber for different input patterns (e.g. different focus positions on the fiber input facet and/or different input wavelengths). During the pre-calibration, the background signal corresponding to each speckle pattern can also be recorded. It will be appreciated that the pre-calibration procedure for compressive endo-microscopy doesn't require additional calculations and can thus be more straightforward than needed for raster scan multimode fiber endo-microscopy.

After the pre-calibration, the sample was placed against the output facet of the multimode fiber and the calibration part of the setup may be removed. The sample image was acquired in an endoscopy configuration by sequentially applying the same phase masks as during the calibration procedure and detecting the total fluorescent signal. An example of raw data averaged over three measurements after background subtraction is presented in FIG. 9(c). Error bars represent the standard deviation. The Signal to Noise Ratio was estimated as ≈6. Noise is mostly explained by the lower level of the fluorescent signal approaching the background in comparison to raster scanning endo-microscopy, due to the redistribution of the pump intensity over the full image area. A much smaller dynamic range also plays role. Despite the low signal level and small number of measurements, the image can be recovered very well.

In some embodiments, as used here, to retrieve an image a procedure such as described by E. J. Candes, J. Romberg, and T. Tao [IEEE Trans. Inf. Theory 52, 489 (2006)] can be used. For example, the image retrieval may include calculating the solution to an l₁ minimization problem. For example, the open software algorithm ‘l₁ magic’ from Stanford.edu can be used. In some embodiments, to increase the speed of the calculation, the resolution of the reference speckle patterns can be artificially decreased. In the experiments, the average calculation time was 20 seconds for a 50×50 pixels image and 8 minutes for a 100×100 pixels image. The calculations were done on a standard office PC using a custom algorithm. The retrieved image is shown in FIG. 9(d). It will be appreciated that the standard multimode fiber imaging and novel compressive endo-microscopy provides images of micrometer-size spheres with diffraction-limited resolution. The FWHM of the cross-section is 1.3±0.2 μm for standard endo-microscopy and 1.4±0.2 μm for compressive endo-microscopy.

The imaging speed of compressive micro-endoscopy is significantly higher due to several reasons. Firstly, less measurements are needed to reconstruct a high-resolution image through the chosen fiber probe, e.g. 150 in the experiment. So it may increase the imaging speed (here 23 fold) and increase the speed of pre-calibration (here 18 fold). Secondly, imaging speed is not limited by the speed of a spatial light modulator. In compressive endo-microscopy, e.g. fast galvo mirror systems and/or resonance scanning of the single-mode fiber and/or wavelength variation can be used. It allows to further increase the imaging speed to milliseconds per frame. It can be used for many applications such as imaging of neuron activity with fast potential sensitive dyes.

Without being bound by theory, it will be appreciated that in compressive sensing, pseudorandom patterns can be used, since they typically are strongly uncorrelated with the common mathematical bases in which natural images have sparse representations. To create pseudorandom illumination patterns e.g. spatial light modulators or random scattering samples can be utilized. In preferred embodiments, as described herein speckle patterns are generated by a multimode fiber. In other or further embodiments, also other types of scattering media may be used.

To analyze the properties of the speckle patterns of a multimode fiber, the correlation coefficient r between two random illumination patterns for a total number of recorded patterns can calculated e.g. using the following equation

$$r = \frac{\sum ( a - \overline{a} ) ( b - \overline{b} )}{\sqrt{\sum {( a - \overline{a} )}^{2} \sum {( b - \overline{b} )}^{2}}}$$

where a and b were images of speckles on the output facet of a multimode fiber, ā and b̅ were their means.

FIG. 10(a) illustrates cross-correlation coefficients of different speckle patterns “Np” generated in a MM fiber for a total 225 patterns.

A total of 225 speckle patterns were created by scanning a focused input beam over 225 points organized in a square lattice (15×15) on the input fiber facet. Correlation coefficients were calculated for all pairs of speckle patterns and the results are presented in FIG. 10(a). It will be appreciated that the correlation between two independent random speckle patterns generated in the multimode fiber is close to 0, confirming their randomness.

FIG. 10(b) illustrates an analysis of the reconstruction success “RS” as a function of the number of measurements “Nm”. Reconstruction success is estimated as cross-correlation coefficient between the image reconstructed with 225 measurements and the image reconstructed with “Nm” measurements, as a function of “Nm”. Bottom dots represent experimental results, top dots represent the numerical simulation based on experimentally measured speckle patterns of a MM fiber. Gray zone represents the area where the quality of reconstruction is substantially preserved.

The measurements may be illustrative of the lower limit of the number of measurements desired for multimode fiber compressive imaging. We repeated the compressive endo-microscopy experiments described above for a number of reference speckle patterns ranging from 40 to 225 with increments of 5. To analyze the noise-free limit of MM fiber compressive imaging we also did numerical experiments. We used an experimentally measured set of speckle patterns on the fiber output facet and simulated the noise-free signal by calculating inner product between the basis set and the object of interest. Then we used the same procedure of l₁ minimization to retrieve an image from the incomplete set of experimental data as well as numerical measurements.

FIGS. 10(c) and (d) illustrate compressive multimode fiber imaging during the scanning of a sample in (c) y- and (d) z-direction with a step size of 5 μm. Scale bars are 5 μm. In the measurements, we used MM fiber compressive endo-microscopy to image a sample were the sample position was changed relative to the fiber output facet in the lateral and axial directions with a step size of 5 μm over 20 μm in total.

We see that the quality of the reconstructed image doesn't depend on the position of the fluorescent sample during lateral scanning. In contrast, we see that the level of signal dramatically decreases when we move a sample away from the fiber facet. The main reason is that the collected signal strongly depends on the distance from the fiber output facet. The image quality and the signal level is maintained only within the first 10 μm. Beyond 15 μm the contribution of the signal is very low as can be seen in FIG. 9(d). Resolution in the axial direction can be further improved by using a fiber with a relatively high NA. We can use this inherent optical sectioning provided by the nature of a fiber for imaging bulk samples. As a result, multimode fiber approach provides a new method for compressive sensing.

To summarize, we showed that the speckles naturally generated in the multimode fiber represent an excellent basis for compressive sensing. We experimentally demonstrated compressive endo-microscopy with a number of measurements much smaller than the number of modes of a multimode fiber, e.g. factor ten or even twenty. Moreover, we show that inherent optical sectioning of a multimode fiber can be used for providing resolution in the axial direction and sectioned imaging of bulk structures. Compressive multimode fiber imaging provides significantly higher speed, keeps diffraction limited resolution and doesn't require complex wavefront shaping offering a unique tool for endo-microscopy.

In interpreting the appended claims, it should be understood that the word “comprising” does not exclude the presence of other elements or acts than those listed in a given claim; the word “a” or “an” preceding an element does not exclude the presence of a plurality of such elements; any reference signs in the claims do not limit their scope; several “means” may be represented by the same or different item(s) or implemented structure or function; any of the disclosed devices or portions thereof may be combined together or separated into further portions unless specifically stated otherwise. Where one claim refers to another claim, this may indicate synergetic advantage achieved by the combination of their respective features. But the mere fact that certain measures are recited in mutually different claims does not indicate that a combination of these measures cannot also be used to advantage. The present embodiments may thus include all working combinations of the claims wherein each claim can in principle refer to any preceding claim unless clearly excluded by context.

1. An imaging system comprising
a light source;
a spatial light modulator, multimode waveguide, or other type of scattering medium, configured to receive input light from the light source, and generate a respective pseudorandom illumination pattern based on the input light; and
a controller configured to
receive calibration data relating a predetermined set of input characteristics of the input light with a corresponding set of spatial distributions of the respective pseudorandom illumination pattern;
receive a set of intensity measurements of a light signal from a sample illuminated by different pseudorandom illumination patterns according to the set of predetermined spatial distributions; and
calculate a spatially resolved image of the sample based on the intensity measurements and calibration data, using a compressive sensing algorithm.

2. The system according to claim 1, further comprising
a multimode waveguide configured to receive the input light from the light source into its proximal side and output a corresponding speckle pattern as the respective pseudorandom illumination pattern based on the input light out of its distal side for illuminating the sample to be imaged; and
a single-mode waveguide connected to the multimode waveguide for coupling the input light from the light source to the multimode waveguide,
wherein the multimode waveguide has a flexural rigidity higher than that of the single-mode waveguide by at least a factor ten, and the multimode waveguide has a relatively short length less than ten centimeters and the single-mode waveguide has a relatively long length at least ten times longer than the multimode waveguide.

3. The system according to claim 1, wherein the predetermined set of input characteristics comprises a set of different wavelengths of the input light.

4. The system according to claim 3, wherein the light source is a broad band light source configured to generate the input light over a range of different wavelengths.

5. The system according to claim 4, comprising a light detector with a spectral resolving element for measuring an intensity of the light signal as a function of wavelength, and a light sensor with a plurality of sensor elements for simultaneously measuring spectral intensities of the light signal.

6. The system according to claim 5, wherein the controller is configured to calculate the spatially resolved image based on one or more shots of the broad band light source and corresponding measurements of the spectral intensities of the light signal.

7. The system according to claim 1, comprising a multi-clad fiber formed by at least a fiber core with a first fiber cladding around the fiber core, and a second fiber cladding surrounding the first fiber cladding, wherein the fiber core forms the single-mode waveguide for the input light and the first fiber cladding forms a return path for the measured light signal, wherein both the fiber core and first fiber cladding are connected to couple the input light into, and the signal light out of, the multimode optical fiber.

8. (canceled)

9. The system according to claim 1, wherein the multimode waveguide is formed by a multimode optical fiber held fixated by a rigid mantle.

10. The system according to claim 1, wherein the multimode waveguide is arranged in a hollow epidural needle.

11. The system according to claim 1, wherein an output of a single mode fiber forming the single-mode waveguide is fused to proximal side of the multimode waveguide formed by a multimode optical fiber.

12. The system according to claim 1, wherein a position of an end of the single-mode waveguide, is varied with respect to the proximal side of the multimode waveguide to provide a set of different input characteristics.

13. A method comprising
receiving calibration data relating a predetermined set of input characteristics of input light with a corresponding set of spatial distributions of a respective pseudorandom illumination pattern generated by a spatial light modulator, multimode waveguide, or other type of scattering medium, based on the input characteristics;
receiving a set of intensity measurements of a light signal from a sample illuminated by different pseudorandom illumination patterns according to the set of predetermined spatial distributions; and
calculating a spatially resolved image of the sample based on the intensity measurements and calibration data, using a compressive sensing algorithm.

14. A non-transitory computer readable medium storing program instructions which when executed by a computer cause the computer to perform a method comprising
receiving calibration data relating a predetermined set of input characteristics of input light with a corresponding set of spatial distributions of a respective pseudorandom illumination pattern generated by a spatial light modulator, multimode waveguide, or other type of scattering medium, based on the input characteristics;
receiving a set of intensity measurements of a light signal from a sample illuminated by different pseudorandom illumination patterns according to the set of predetermined spatial distributions; and
calculating a spatially resolved image of the sample based on the intensity measurements and calibration data, using a compressive sensing algorithm.

15. (canceled)

16. The method according to claim 13, wherein the calibration data comprises a set of coefficients of a neural network, wherein the neural network is trained using the predetermined set of input characteristics of input light and respective pseudorandom illumination patterns according to the set of predetermined spatial distributions, wherein the spatially resolved image is calculated based on the coefficients.

17. The method according to claim 13, wherein the input characteristics comprise a set of different wavelengths of the input light, and the light signal from the sample is spectrally resolved to determine the set of intensity measurements.
